(19) United States
(12) Patent Application Publication
(10) Pub. No.: US 2023/0371794 A1
(43) Pub. Date: Nov. 23, 2023

(54) REUSABLE IRRIGATION SHEATH FOR AN ENDOSCOPE

(71) Applicant: Henke-Sass, Wolf GmbH, Tuttlingen (DE)

(72) Inventors: Ina DOMINKE, Bad Duerrheim (DE); Daniel SEEH, Immendingen (DE)

(21) Appl. No.: 18/199,356

(22) Filed: May 18, 2023

(30) Foreign Application Priority Data
May 19, 2022 (DE) 102022112589.0

(51) Int. Cl. A61B 1/00

(52) U.S. Cl. CPC A61B 1/00135; A61B 1/00142

(57) ABSTRACT

A reusable irrigation sheath for an endoscope includes a sheathing tube connected to an end portion, a fixing unit formed at the end portion, and a rinsing port fluid-connected to the sheathing tube. The irrigation sheath includes an endoscope channel extending through the end portion and the sheathing tube. Once the endoscope shaft has been introduced into the endoscope channel and has reached the work position, the irrigation sheath is fixed to the endoscope via the fixing unit so that the distal end of the endoscope shaft rests against an axial stop and the radial position of the endoscope shaft in the sheathing tube is specified by at least two radial stops such that a rinsing channel located within the sheathing tube and in fluid connection with the rinsing port extends up to the distal end of the sheathing tube.

### PRIORITY

This application claims the benefit of German Patent Application No. 10 2022 112 589.0, filed May 19, 2022, which is hereby incorporated herein by reference in its entirety.

### FIELD

The present invention relates to a reusable irrigation sheath for an endoscope comprising a main part and a rigid endoscope shaft which extends away from the main part and which comprises a cover glass at its distal end distant from the main part.

### BACKGROUND

Previously known irrigation sheaths are in the form of a disposable product which should be disposed after use.

### SUMMARY

It is an object herein to provide an improved irrigation sheath for an endoscope.

In certain embodiments, the reusable irrigation sheath (or reusable rinsing sheath) is provided for an endoscope comprising a main part and a rigid endoscope shaft which extends away from the main part and which comprises a cover glass at its distal end distant from the main part. The irrigation sheath comprises a proximal end portion comprising a proximal end and a distal end, a sheathing tube connected to the distal end of the proximal end portion, a fixing unit formed at the proximal end portion, and a rinsing port fluid-connected to the sheathing tube. The rinsing port can preferably be formed at the proximal end portion. Further, the irrigation sheath comprises an endoscope channel extending through the proximal end portion and the sheathing tube from the proximal end of the proximal end portion such that, with its distal end, the endoscope shaft is introducible into the endoscope channel via the proximal end of the proximal end portion until a work position for the endoscope has been reached. At its distal end distant from the proximal end portion, the sheathing tube to this end comprises an axial stop, which limits the maximum entry depth of the endoscope shaft into the endoscope channel, and at least two radial stops, which specify or define the radial position of the endoscope shaft in the sheathing tube. Once the endoscope shaft has been introduced into the endoscope channel and has reached the work position, the irrigation sheath is releasably fixed or releasably attached to the endoscope by means of the fixing unit so that the distal end of the endoscope shaft rests against the axial stop and the radial position of the endoscope shaft in the sheathing tube is specified by the at least two radial stops such that a rinsing channel located within the sheathing tube and in fluid connection with the rinsing port extends up to the distal end of the sheathing tube. Hence, a fluid (or rinsing fluid) supplied via the rinsing port can flow through the rinsing channel and over the cover glass positioned at the distal end of the sheathing tube in the work position, in order to clean the said cover glass. In particular, this can be carried out when the endoscope is used as intended.

The proximal end portion, the rinsing port, and the sheathing tube can each be made of stainless steel. This enables reusability of the rinsing sheath.

In particular, the sheathing tube can be adhesively bonded and/or welded to the proximal end portion. Similarly, the rinsing port can be adhesively bonded and/or welded to the proximal end portion.

In particular, the fixing unit can be designed such that in the work position the axial stop presses against the distal end of the endoscope shaft with a predetermined force so that there is a predetermined clamping effect in the axial direction.

The rinsing port can open into the endoscope channel at an opening, wherein a ring groove with an inserted ring seal can be provided at a position located closer to the proximal end of the proximal end portion than the position of the opening. The ring groove with the inserted ring seal can seal the endoscope channel when the endoscope has been introduced.

By way of example, the ring seal can be in the form of an O-ring. Further, the ring seal may serve to inhibit axial movement between the endoscope shaft and the rinsing sheath.

On account of the clamping effect provided by the fixing unit, the ring groove and the ring seal may be designed such that there is a comparatively small inhibitory effect. For example, for an endoscope shaft with an external diameter of 4 mm in the dry state, a force ranging from 3.5 N to 5.8 N may be sufficient for guiding the endoscope shaft through the distal end in the endoscope channel and past the seal in the process.

As a result of this small inhibitory effect of the ring seal, it is easily possible to place the irrigation sheath onto, and remove it again from, an endoscope shaft several hundred times. If the ring groove and the seal were to be dimensioned so that larger forces are necessary to introduce the endoscope shaft, then this would lead relatively quickly to the shearing off or damage to the seal, with the result that reusability could not be ensured.

Further, the fixing unit may comprise a receiving portion in which a part of the endoscope (for example the main part) is seated or against which a part of the endoscope rests once the endoscope shaft has been introduced into the endoscope channel and has reached the work position. In particular, the receiving portion may comprise a concave basic shape.

The fixing unit can be formed in one piece with the proximal end portion.

Further, the at least two radial stops can be formed in one piece with the sheathing tube. In particular, the at least two radial stops can project into the interior of the sheathing tube and can be formed by a thickening in the wall of the sheathing tube so that the outer contour of the sheathing tube is constant over the entire length (with the exception of the region of the axial stop) and consequently also constant in the entire region of the radial stops. Hence, the sheathing tube may comprise a continuously smooth outer surface, which is advantageous for autoclaving purposes and hence for the reusability of the rinsing sheath.

The at least two radial stops may have a rounded-off form as seen in the longitudinal direction of the sheathing tube. This makes it possible to insert the endoscope shaft and withdraw the endoscope shaft without problems since jamming of or damage to the distal end of the endoscope shaft can be avoided as a result of the rounded-off form in the longitudinal direction.

Further, the axial stop may also be formed in one piece with the sheathing tube. The axial stop can also be formed by a thickening in the wall of the sheathing tube which protrudes into the interior of the sheathing tube. Consequently, a smooth continuous outer surface of the sheathing tube can also be provided in the region of the axial stop.

The two radial stops and the axial stop can be located at the corners of an isosceles triangle in a view on the distal end of the sheathing tube.

Further, it is possible that the at least two radial stops (preferably exactly two radial stops) are arranged so that the introduced endoscope shaft is tilted vis-à-vis the sheathing tube, with the pivot point of the tilt being located in the region of the proximal end of the endoscope channel. The endoscope shaft is preferably arranged in centered fashion in the region of the pivot point.

However, it is also possible that three radial stops (preferably exactly three radial stops) are provided and are arranged such that the introduced endoscope shaft is arranged coaxially with the sheathing tube so that the rinsing channel comprises a ring-shaped cross section.

The two radial stops may be spaced apart by 90°, in particular, in the circumferential direction. The spacing may also be greater and for example be 120°, 130°, or 140°. Further, it is also possible for the spacing to be less, for example 80°, 70°, 60°, or 45°.

Three radial stops which are preferably spaced equidistantly apart from one another in the circumferential direction may be provided in the rinsing sheath.

The inner diameter of the sheathing tube is preferably greater than the outer diameter of the endoscope shaft. In particular, the inner diameter of the sheathing tube may be greater than the outer diameter of the endoscope shaft by 1% to 10% (e.g., 2%, 3%, 4%, 5%, 6%, 7%, 8%, 9%, 9.5%, 9.6%, 9.7%, or 9.8%).

By way of example, the radial stops may protrude from the inner side of the wall of the sheathing tube by a value ranging from 0.10 to 0.30 mm. This can provide a rinsing channel which comprises a maximum radial extent ranging from 0.10 to 0.30 mm.

Further, a plurality of axial stops which are spaced apart from one another in the circumferential direction may be provided. In particular, the plurality of axial stops may be spaced equidistantly apart from one another in the circumferential direction.

The sheathing tube can be in the form of a single-walled sheathing tube. The wall thickness of the wall may be constant in particular, with the exception of the regions in which the stops are formed.

Further, the sheathing tube can be in the form of a rigid sheathing tube and/or a sheathing tube extending in a straight line.

The sheathing tube can have a hollow cylindrical form. The sheathing the tube can comprise a round, oval or ring-shaped outer and/or inner cross section.

In particular, the distal end of the sheathing tube is designed (especially on account of the axial stop) so that the distal end of the endoscope shaft cannot be introduced into the endoscope channel beyond the distal end of the sheathing tube. By way of example, this can reliably prevent a non-intended introduction of the endoscope shaft into the rinsing sheath.

The sheathing tube can comprise a distal opening at its distal end and a proximal opening at its proximal end distant from the distal end.

Apart from the distal opening, the sheathing tube may comprise no further openings in the region from the proximal end portion to its distal end. In particular, the sheathing tube may comprise no further openings apart from its distal opening and proximal opening.

The sheathing tube (or the wall of the sheathing tube) may comprise a continuously smooth outer surface in the region from the proximal end portion to its distal end. In particular, such a continuously smooth outer surface is understood to mean an outer surface which, apart from the distal opening and proximal opening, comprises no further openings that extend into the interior of the sheathing tube. Such a continuously smooth outer surface is further understood to mean in particular an outer surface which has no depressions or the like and thus e.g. no edges or the like.

In particular, the irrigation sheath can be designed so that it is autoclavable (especially as a whole).

Moreover, an endoscope together with the reusable irrigation sheath is provided.

In particular, the endoscope can be configured as a sinuscope.

It is understood that the features mentioned above and the features yet to be explained hereinafter can be used not only in the combinations specified but also in other combinations or on their own, without departing from the scope of the present invention.

The invention will be explained in even more detail hereinafter on the basis of exemplary embodiments, with reference being made to the attached drawings which likewise disclose features essential to the invention. These exemplary embodiments only serve illustrative purposes and should not be construed as limiting. By way of example, a description of one exemplary embodiment comprising a multiplicity of elements or components should not be construed as meaning that all of these elements or components are required for implementation purposes. Rather, other exemplary embodiments may also contain alternative elements and components, fewer elements or components, or additional elements or components. Elements or components of different exemplary embodiments can be combined with one another provided nothing else is specified. Modifications and variations described for one of the exemplary embodiments may also be applicable to other exemplary embodiments. To avoid repetition, similar or corresponding elements are denoted by the same reference sign throughout the figures and are not explained multiple times.

### BRIEF DESCRIPTION OF THE DRAWINGS

FIG. 1 is a sectional view of an embodiment of the irrigation sheath according certain embodiments of the invention.

FIG. 2 is a plan view of the irrigation sheath of FIG. 1.

FIG. 3 is a side view of an endoscope.

FIG. 4 is a side view of the irrigation sheath with an inserted endoscope according to FIG. 3.

FIG. 5 is a magnified sectional illustration of the distal end of the sheathing tube.

FIG. 6 is a magnified sectional illustration of the distal end of the sheathing tube with an inserted endoscope shaft.

FIG. 7 is a perspective illustration of the distal end of the sheathing tube.

FIG. 8 is a further perspective illustration of the distal end of the sheathing tube.

FIG. 9 is a view of the proximal end portion on the distal end of the sheathing tube.

FIG. 10 is a sectional view of the sheathing tube in the region of the radial stops.

FIG. 11 is a sectional view of the distal end of the sheathing tube of a further embodiment of the irrigation sheath with an inserted endoscope shaft.

FIG. 12 is a perspective view of the distal end of the sheathing tube according to FIG. 11.

FIG. 13 is a sectional view of the sheathing tube according to FIG. 11 in the region of the radial stops.

FIG. 14 is a sectional view of the distal end of the sheathing tube of a further embodiment of the irrigation sheath with an inserted endoscope shaft.

FIG. 15 is a perspective view of the distal end of the sheathing tube according to figure.

FIG. 16 is a sectional view of the sheathing tube according to FIG. 14 in the region of the radial stops.

FIG. 17 is a sectional view of the distal end of the sheathing tube in a further embodiment of the rinsing sheath with an inserted endoscope shaft.

FIG. 18 is a perspective view of the distal end of the sheathing tube according to FIG. 17.

FIG. 19 is a sectional view of the sheathing tube of FIG. 17 in the region of the radial stops.

### DETAILED DESCRIPTION

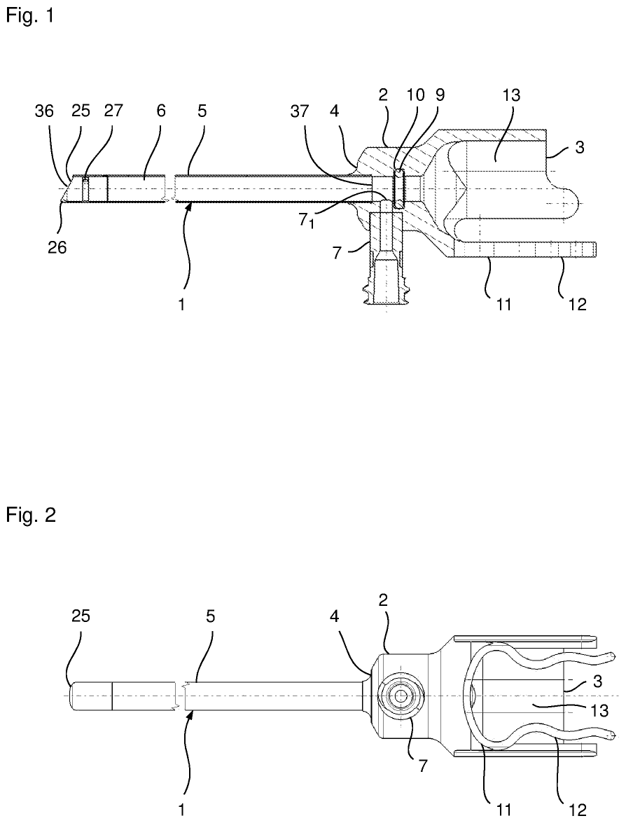

In the embodiment of the reusable irrigation sheath 1 for an endoscope shown in FIGS. 1 and 2, the irrigation sheath 1 comprises a proximal end portion 2 with a proximal end 3 and a distal end 4. A sheathing tube 5, for example of hollow cylindrical shape with a circular cross section, is formed at the distal end 4. The sheathing tube 5 comprises a distal opening 36 and a proximal opening 37, and may be adhesively bonded and/or welded to the end portion 2.

As may be gathered from FIG. 1 in particular, the irrigation sheath 1 comprises an endoscope channel 6 extending through the end portion 2 and the sheathing tube 5 from the proximal end 3 of the end portion 2.

Further, a rinsing port 7, which is fluid-connected to the endoscope channel 6, is formed at the proximal end portion 2. The rinsing port 7 may be adhesively bonded and/or welded to the end portion 2. As is evident from FIG. 1 in particular, a seal 9 (for example, an O-ring 9) is seated in a ring groove 10 which is formed closer to the proximal end 3 than the opening 71 of the rinsing port 7 into the endoscope channel 6.

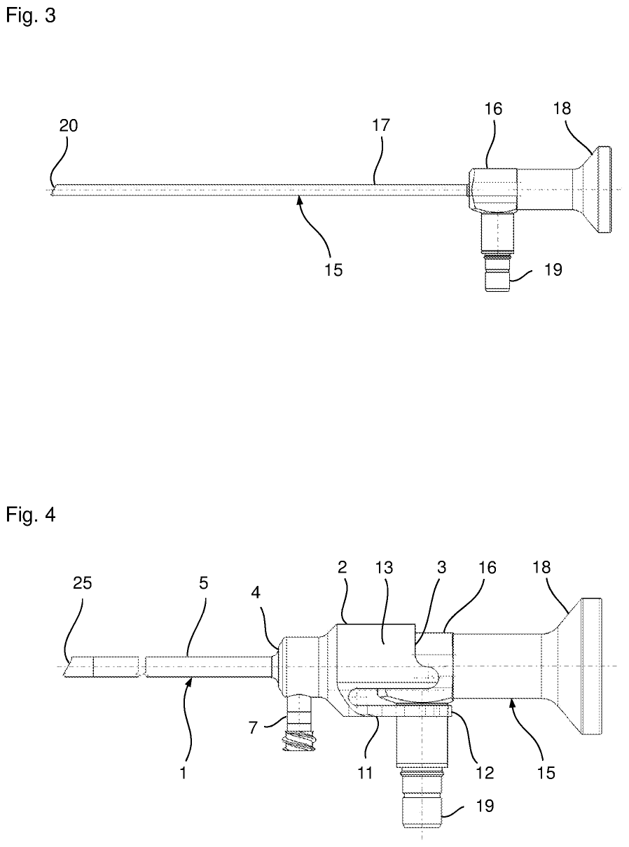

As will be described in detail hereinafter, the proximal end portion 2 further comprises a fixing unit 11, by means of which the irrigation sheath 1 is detachably attachable to an endoscope 15, for example as shown in FIG. 3. In this case, the fixing unit 11 comprises an elastic clamp 12, for example as is evident in the plan view according to FIG. 2, and a receiving portion 13. In this case, the clamp 12 is formed in one piece with the remaining proximal end portion 2. The proximal end portion 2, the sheathing tube 5, and the rinsing port 7 are made from stainless steel, with the result that the irrigation sheath 1 is autoclavable as a whole.

The endoscope 15 for which the irrigation sheath 1 is designed comprises a main part 16, a rigid endoscope shaft 17 extending away from the main part 16, an eyepiece 18 provided on the main part 16, and a light-guide connector 19 (as illustrated in FIG. 3). The endoscope 15 further comprises an observation optical unit (not shown) which extends through the endoscope shaft 17, with the distal end 20 of the endoscope shaft 17 being terminated by a cover glass 21 (FIG. 6). Consequently, an observer can observe, in a manner known per se, the region in front of the distal end 20 through the eyepiece 18. To this end, a light source can be connected, in particular to the light-guide connector 19, with the result that the region in front of the distal end 20 is simultaneously also illuminated by way of the endoscope shaft 17. The endoscope 15 shown here is a sinuscope with a viewing direction of 30° with respect to the longitudinal axis of the endoscope shaft 17.

The irrigation sheath 1 is designed so that the inner diameter of the endoscope channel 6 and in particular the inner diameter of the sheathing tube 5 are greater than the outer diameter of the endoscope shaft 17. Thus, the inner diameter of the sheathing tube can be for example 1%-10% greater than the outer diameter of the endoscope shaft 17. By way of example, if the outer diameter of the endoscope shaft 17 is 3.1 mm, then the inner diameter of the sheathing tube 5 can be 3.4 mm. By way of example, if the outer diameter of the endoscope shaft 17 is 4.0 mm, then the inner diameter of the sheathing tube 5 can be 4.2 mm.

The irrigation sheath 1 can be pushed onto the endoscope shaft 17 over the distal end 20 of the endoscope shaft 17 until the main part 16 is seated in or rests against the receiving portion 13, and the clamp 12 of the fixing unit 11 resiliently engages around the light-guide connector 19, as depicted schematically in the side view according to FIG. 4.

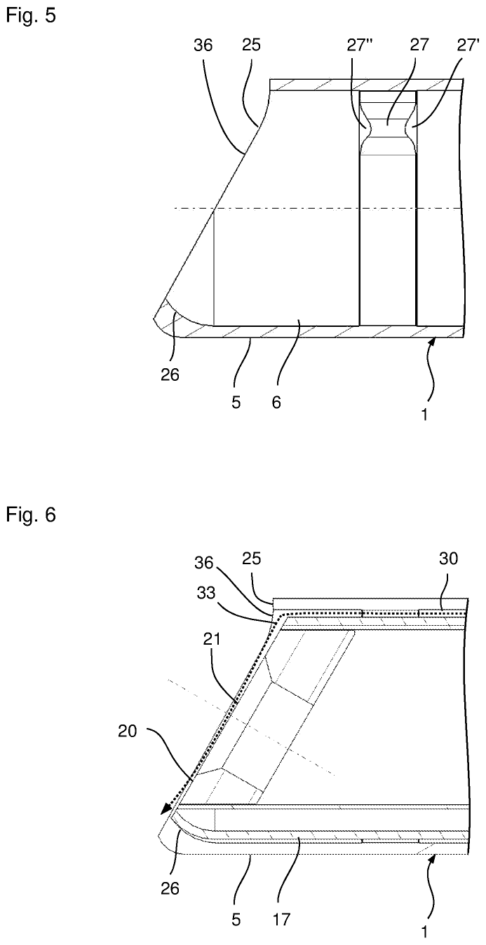

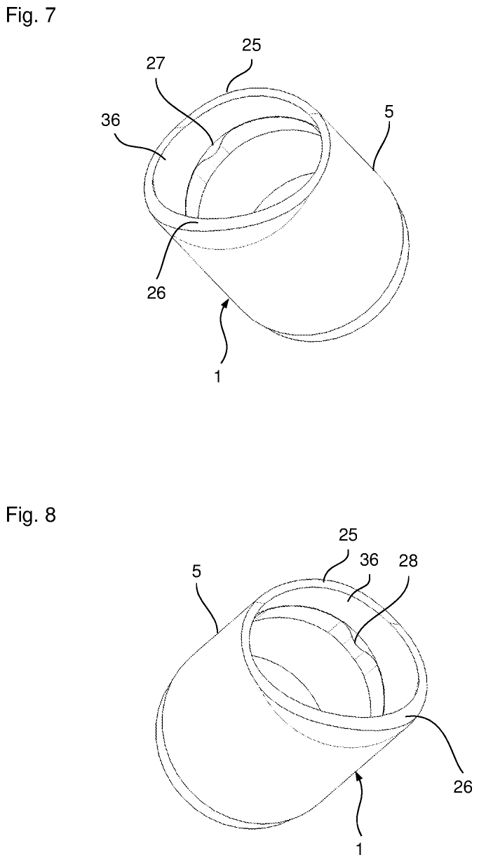

As is evident from the illustrations according to FIGS. 5 to 10, for example, the design of irrigation sheath 1 at its distal end 25 is such that it comprises a distal stop 26 for the distal end 20 of the endoscope shaft 17. Further, the wall of the sheathing tube 5 comprises two radial stops 27, 28 in the region of the distal end of the irrigation sheath 1.

The dimensions of the irrigation sheath 1 are chosen so that, in the work position shown in FIG. 4, the distal end 20 of the endoscope shaft 17 rests against the distal stop 26. A certain amount of force which presses the distal stop 26 against the distal end 20 of the endoscope shaft 17 is exerted in the process on account of the irrigation sheath 1 being fixed to the endoscope 15 by means of the fixing unit 11. Consequently, the axial position of the endoscope shaft 17 is secured in the work position.

The radial position of the endoscope shaft 17 is also specified by the two radial stops 27 and 28, against which the endoscope shaft 17 rests. Here, this radial position is specified such that the endoscope shaft 17 and the sheathing tube 5 are not aligned coaxially with one another, but are tilted with respect to one another. Thus, the radial stops 27, 28 press the endoscope shaft 17 to the side of the sheathing tube 5 opposite to the radial stops 27, 28, while the endoscope shaft 17 in the region of the seal 9 is positioned in the endoscope channel 6 in centered fashion. Hence, the pivot point of the tilt of the endoscope shaft 17 is located in the region of the seal 9 (or in the region of the proximal end 3 of the end portion 2 or in the region of the receiving portion 13). The fixing unit 11 is designed to enable such a tilt.

On account of this tilt and the inner diameter of the sheathing tube 5, which is greater than the outer diameter of the endoscope shaft 17, a rinsing channel 30 is formed between the endoscope shaft 17 and the wall of the sheathing tube 5, and the said rinsing channel extends from the opening 71 of the rinsing port 7 to the distal end 25 of the sheathing tube 5. On account of the centered positioning of the endoscope shaft 17 in the sheathing tube 5 present in the region of the opening 71, the rinsing channel 30 comprises a ring-shaped portion surrounding the endoscope shaft 17 at that location. In the direction toward the distal end 25 of the irrigation sheath 1, the rinsing channel 30 is formed ever more predominantly by a portion 32 with a ring segment-shaped cross section between the two radial stops 27 and 28, as illustrated in FIG. 10, on account of the tilt of the endoscope shaft 17 in the sheathing tube 5. The portion 32 comprises a maximal radial extent D which for example ranges between 0.10 and 0.20 mm for endoscopes 15 with an outer diameter of the endoscope shaft 17 of 4 mm. In the case of a irrigation sheath 1 for endoscopes 15 with an outer diameter of the endoscope shaft 17 of 3.1 mm, the maximal radial extent may range from 0.20 to 0.30 mm. What this achieves is that the majority of the rinsing fluid emerges through this portion 32 (via the distal opening 36) at the distal end 25 of the irrigation sheath 1. Hence, rinsing fluid supplied via the rinsing port 7 will flow along the rinsing channel 30 to the distal end 25 of the sheathing tube 5 and will reliably flow over the cover glass 21 of the endoscope shaft 17 at said location, as indicated by arrow 33 in FIG. 6. In this way, it is possible to ensure cleaning of the cover glass 21 by means of the rinsing fluid, for example during the intended use of the endoscope 15.

The seal 9 serves to seal the rinsing channel 30 vis-à-vis the proximal end 3. Further, the seal 9 also serves to inhibit axial movement between the endoscope shaft 17 and the irrigation sheath 1. Since the irrigation sheath 1 is already fixed to the endoscope 15 to a sufficient extent on account of the fixing unit 11 in conjunction with the distal stop 26, the ring groove 10 and the seal 9 can be designed so that the inhibitory effect of the seal 9 is relatively small. For an endoscope 15 with an outer diameter of the endoscope shaft 17 of 4 mm, a force ranging from 3.5 N to 5.8 N will be required, for example in the dry state, to insert the endoscope shaft 17 into the endoscope channel 6 through the proximal end 3 and to guide the said endoscope shaft past the seal 9 in the process. This is accompanied by the advantage of it readily being possible to place the irrigation sheath 1 onto and withdraw the latter from an endoscope 15 100 to 500 times, and also to subject the said irrigation sheath 1 to autoclaving in the intervening period of time. Hence the irrigation sheath 1 is reusable.

The two radial stops 27 and 28 are designed to be cam-shaped here. In particular, the radial stops 27, 28 can be formed by local thickening in the wall of the sheathing tube 5, with the result that the radial stops 27, 28 are a part of the wall of the sheathing tube 5 and hence formed in one piece with the sheathing tube 5. Further, the outer contour of the sheathing tube 5 is the same in cross section over the entire length of the said sheathing tube 5 (with the exception of the region with the axial stop 26). Hence, the outer side of the sheathing tube 5 is a smooth, continuous area which, for example, is advantageous for autoclaving and hence also for the reusability. The distal stop 26 can also be formed in one piece with the sheathing tube 5. Here, too, local thickening or appropriate shaping is possible.

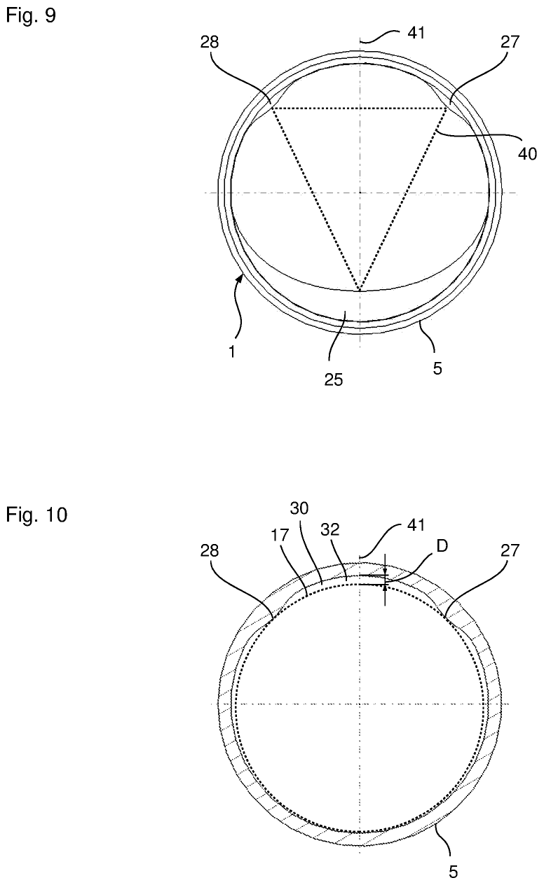

As may be gathered from the view of the distal end 25 of the sheathing tube 5 in FIG. 9 and from the sectional view in FIG. 10, the two radial stops 27, 28 (or the two cams 27, 28) are spaced apart from one another by 90° in the circumferential direction. However, the spacing may also be greater and for example be 120°, 130°, or 140°. Further, it is also possible for the spacing to be less, and for example be 80°, 70°, 60°, or 45°. In the circumferential direction, the axial stop 26 comprises the same distance in terms of magnitude to both the first and the second radial stop 27 and 28. In the exemplary embodiment shown here, it is 135°. Consequently, in the view according to FIG. 9, the stops 26 to 28 are arranged at the corners of an isosceles triangle 40, which has been plotted only for illustrative purposes using dashed lines in FIG. 9. The two radial stops 27 and 28 are arranged mirror-symmetrically with respect to a diameter 41, with the axial stop 26 being located on this diameter 41.

As may be gathered from the illustration in FIG. 5 in particular, the radial stop 27 is designed such that it is rounded-off in the longitudinal direction of the sheathing tube 5 and consequently comprises rounded-off portions 27′ and 27″. The radial stop 28 is designed such that it is rounded-off in the longitudinal direction of the sheathing tube 5, in the same way as the radial stop 27. As a result, it is possible to ensure that the distal end 20 of the endoscope shaft 17 is guided by the portion 27′ and hence routed over the cam 27 when the endoscope shaft 17 is introduced. The portion 27″ serves to prevent jamming or wedging during the withdrawal. Consequently, the endoscope shaft 17 can easily be introduced into the sheathing tube 5 and easily be pulled out of the sheathing tube 5 again.

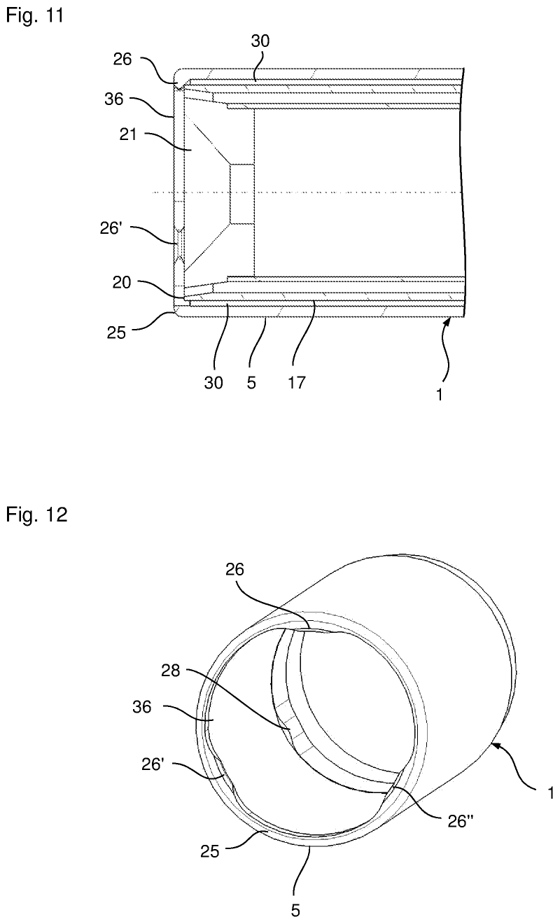

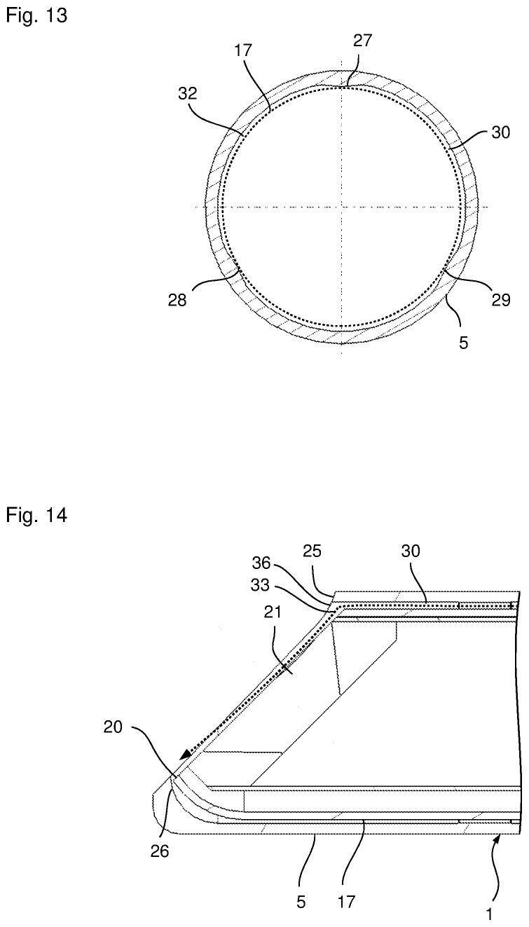

FIGS. 11 to 13 show a modification of the irrigation sheath 1. In this modification, the irrigation sheath 1 is designed for an endoscope 15 with a viewing direction of 0°, with three radial stops 27, 28, 29 being provided in this case such that the endoscope shaft 17 in the work position is positioned coaxially within the sheathing tube 5. The three radial stops 27 to 29 are each spaced apart from one another by 120° in the circumferential direction and are each designed such that they are rounded-off in the longitudinal direction of the sheathing tube 5. Three distal stops 26, 26′, and 26″ are provided in the same way and are likewise spaced apart from one another by 120° in the circumferential direction. Hence, a rinsing channel 30 with a substantially ring-shaped cross section is present, the latter only being interrupted in cross section by the radial stops 27-29 in the portion 32 of the radial stops 27-29, as illustrated schematically in FIG. 13. Therefore, the rinsing channel 30 may also be said to comprise a ring-shaped cross section.

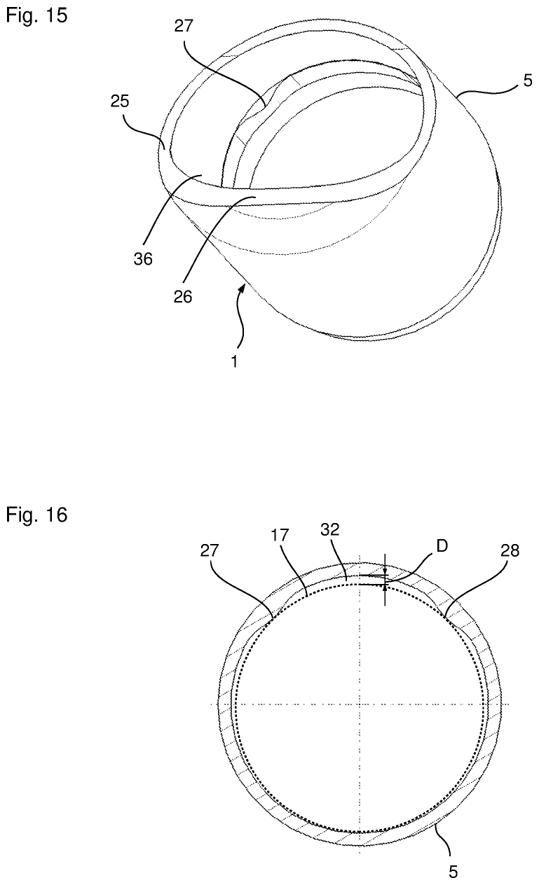

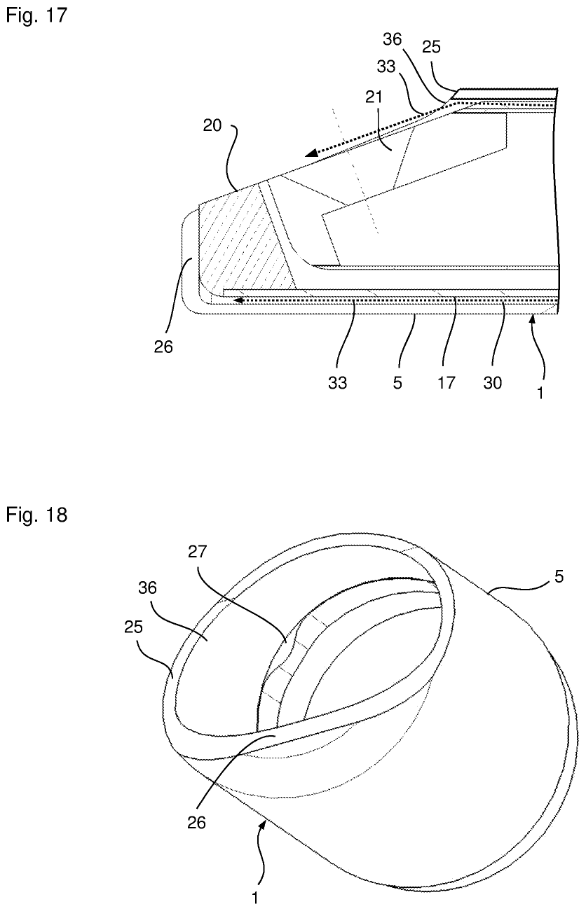

FIGS. 14 to 16 illustrate a variant of the irrigation sheath 1 for an endoscope 15 with a viewing direction of 45°. As may be gathered from the drawings, two radial stops 27 and 28 are provided such that the endoscope shaft 17 extends not coaxially with but tilted to the sheathing tube 5, and so a rinsing channel 30 like in the embodiment according to FIGS. 1-10 is present. Further, a radial stop 26 is provided.

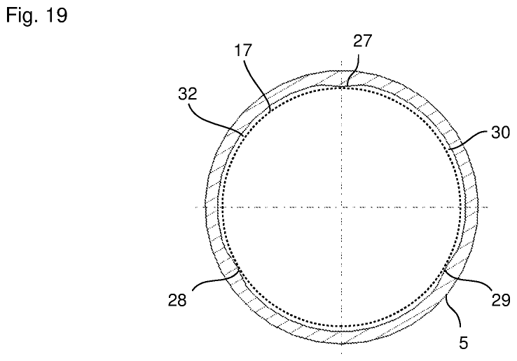

FIGS. 17 to 19 show an exemplary embodiment of the irrigation sheath 1 which is designed for an endoscope 15 with a viewing direction of 70°. Three radial stops 27 to 29 are provided in this case, as illustrated in FIG. 19. Further, a distal stop 26 is provided, with the result that the position of the endoscope shaft 17 in the work position is predefined. A rinsing channel 30 with a ring-shaped cross section is once again present as a result of the three radial stops 27-29.

What is claimed is:

1. A reusable irrigation sheath for an endoscope, comprising:
a main part; and
a rigid endoscope shaft which extends away from the main part and which comprises a cover glass at a distal end thereof distant from the main part,
wherein the irrigation sheath comprises:
a proximal end portion comprising a proximal end and a distal end,
a sheathing tube connected to the distal end of the proximal end portion,
a fixing unit disposed at the proximal end portion,
a rinsing port fluid-connected to the sheathing tube, and
an endoscope channel extending through the proximal end portion and the sheathing tube from the proximal end of the proximal end portion such that, with its distal end, the endoscope shaft is introducible into the endoscope channel via the proximal end of the proximal end portion until a work position for the endoscope has been reached,
wherein, at its distal end distant from the proximal end portion, the sheathing tube comprises:
an axial stop, which limits a maximum insertion depth of the endoscope shaft into the endoscope channel, and
at least two radial stops, which specify the radial position of the endoscope shaft in the sheathing tube,
wherein, once the endoscope shaft has been introduced into the endoscope channel and has reached the work position, the irrigation sheath is fixed to the endoscope via the fixing unit so that the distal end of the endoscope shaft rests against the axial stop and the radial position of the endoscope shaft in the sheathing tube is specified by the at least two radial stops such that a rinsing channel located within the sheathing tube and in fluid connection with the rinsing port extends up to the distal end of the sheathing tube.

2. The irrigation sheath of claim 1, wherein the proximal end portion, the rinsing port, and the sheathing tube are each formed of stainless steel.

3. The irrigation sheath of claim 1,
wherein the rinsing port opens into the endoscope channel at an opening, and
wherein a ring groove with an inserted ring seal for sealing the endoscope channel when the endoscope shaft has been introduced is provided at a position located closer to the proximal end of the proximal end portion.

4. The irrigation sheath of claim 1, wherein the fixing unit is formed in one piece with the proximal end portion.

5. The irrigation sheath of claim 1, wherein the at least two radial stops are formed in one piece with the sheathing tube.

6. The irrigation sheath of claim 1,
wherein the at least two radial stops project into the interior of the sheathing tube and are formed by a thickening in the wall of the sheathing tube so that the sheathing tube comprises a continuously smooth outer surface, and
wherein the at least two radial stops have a rounded-off form in the longitudinal direction of the sheathing tube.

7. The irrigation sheath of claim 1, wherein the fixing unit is configured such that in the work position the axial stop presses against the distal end of the endoscope shaft with a predetermined force so that there is a predetermined clamping effect in the axial direction.

8. The irrigation sheath of claim 1, wherein the axial stop is formed in one piece with the sheathing tube.

9. The irrigation sheath of claim 1, wherein the at least two radial stops and the axial stop are located at corners of an isosceles triangle in a view on the distal end of the sheathing tube.

10. The irrigation sheath of claim 1, wherein the at least two radial stops are arranged so that the introduced endoscope shaft is tilted vis-à-vis the sheathing tube, with the pivot point of the tilt being located in the region of the proximal end of the proximal end portion.

11. The irrigation sheath of claim 1, wherein three radial stops are provided and are arranged such that the introduced endoscope shaft is positioned coaxially within the sheathing tube.

12. The irrigation sheath of claim 1, wherein three radial stops are provided and are spaced equidistantly apart from one another in a circumferential direction.

13. The irrigation sheath of claim 1, wherein a plurality of axial stops which are spaced apart from one another in the circumferential direction are provided.

14. The irrigation sheath of claim 1, wherein the sheathing tube is a single-walled sheathing tube.

15. The irrigation sheath of claim 1,
wherein the sheathing tube comprises a distal opening at its distal end, and
wherein, apart from the distal opening, the sheathing tube comprises no further openings in the region from the proximal end portion to its distal end.

16. An endoscope with a reusable irrigation sheath according to claim 1,
wherein the endoscope comprises:
a main part, and
a rigid endoscope shaft which extends away from the main part and which includes a cover glass at a distal end thereof distant from the main part.
